(19) United States
(12) Patent Application Publication
(10) Pub. No.: US 2025/0297433 A1
(43) Pub. Date: Sep. 25, 2025

(54) SOIL MOISTURE CONTENT AND BULK DENSITY MEASUREMENT

(71) Applicant: CATERPILLAR INC., Peoria, IL (US)

(72) Inventor: Mustafa I. ALSALEH, Orland Park, IL (US)

(73) Assignee: Caterpillar Inc., Peoria, IL (US)

(21) Appl. No.: 18/614,108

(22) Filed: Mar. 22, 2024

(51) Int. Cl. E01C 19/28; G01N 27/04

(52) U.S. Cl. CPC E01C 19/288; G01N 27/043

(57) ABSTRACT

In some implementations, a soil compaction measurement controller may measure a soil resistivity during a soil compaction process. The soil compaction measurement controller may query a lookup table that associates the soil resistivity to an estimated density and moisture content. The soil compaction measurement controller may output, to a component of a machine, a control signal to control an operation of the machine, during the soil compaction process, in accordance with the estimated density and moisture content.

### TECHNICAL FIELD

The present disclosure relates generally to soil moisture content and bulk density measurement and, for example, to measuring soil moisture content and bulk density in real time during a soil compaction process.

### BACKGROUND

Soil compaction involves the densification of soil to improve its mechanical properties depending on use, such as for supporting building foundations, creating roads, or preparing land for agricultural use. Soil compaction is typically achieved through the use of heavy machinery like rollers, compactors, and tampers, which apply pressure or impact force to the soil, thereby reducing its volume and increasing its load-bearing capacity. The degree of compaction often depends on the type of soil and the intended use of the land. For example, clay-rich soils may require different compaction techniques compared to sandy soils. In agricultural settings, proper soil compaction helps control moisture levels and provide optimal conditions for seed germination and root growth. Over-compaction, however, in agricultural contexts can lead to reduced soil aeration and hinder plant growth.

Soil density and moisture content are two factors that influence the process of soil compaction. Soil density, which refers to the mass of soil per unit volume, indicates how tightly soil particles are packed together. Higher soil density generally implies fewer air gaps between particles, resulting in increased load-bearing capacity and stability. Moisture can make the soil more malleable and easier to compact. However, too little moisture can make the soil stiff and resistant to compaction, while excessive moisture can create a fluid-like state, reducing soil stability and making effective compaction challenging. Measuring soil density and moisture during compaction can be a lengthy and complicated process. For example, soil density and moisture tests often take hours to perform, require special equipment, or require a certified or licensed technician to be present. Further, in some situations, the nature of the soil density and moisture test may involve nuclear materials, which can limit how often the test may be performed at a particular worksite.

U.S. Pat. No. 10,145,837 (the '837 patent) discloses a ground penetrating radar (GPR) system for determining asphalt density and soil moisture. The GPR system has a system controller configured to produce an electromagnetic signal for signal penetration of a pavement material. Further, the GPR system has a frequency modulated continuous wave controller and an ultra wide band (UWB) antenna coupled to the system controller. The UWB antenna transmits the produced electromagnetic signal to the pavement material and receives the electromagnetic signal as a reflection from the pavement material. The system controller receives the electromagnetic signal from the UWB antenna. The GPR system of the '837 patent, however, cannot be used to measure soil density and moisture content in real time during soil compaction. Rather, the GPR system of the '837 patent is intended to be used after pavement material has been applied.

The soil compaction measurement controller of the present disclosure solves one or more of the problems set forth above and/or other problems in the art.

### SUMMARY

A method may include measuring a soil resistivity during a soil compaction process; and querying, during the soil compaction process, a lookup table that associates the soil resistivity to an estimated density and moisture content.

A soil compaction measurement controller may include one or more memories; and one or more processors, communicatively coupled to the one or more memories, configured to: determine a soil resistivity; query a lookup table associating the soil resistivity to an estimated density and moisture content, the lookup table being stored in the one or more memories; and output, to a component of a machine, a control signal to control an operation of the machine in accordance with the estimated density and moisture content.

A soil compactor machine may include a drum; a plurality of electrodes disposed on the drum; a voltmeter electrically connected to at least two of the plurality of electrodes and configured to output a soil voltage measurement; an ammeter electrically connected to at least two of the plurality of electrodes and configured to output a soil current measurement; and a soil compaction measurement controller in communication with the voltmeter and the ammeter, the soil compaction measurement controller being configured to: receive the soil voltage measurement and the soil current measurement; determine an estimated density and moisture content from the soil voltage measurement and the soil current measurement; and control operation of the drum in accordance with the estimated density and moisture content.

### BRIEF DESCRIPTION OF THE DRAWINGS

FIG. 1 is a diagram of an example compactor including a system for measuring soil density and moisture content in real time during soil compaction.

FIG. 2 is a diagram of an example soil measurement device for estimating soil moisture content and bulk density.

FIG. 3 is a flowchart of an example process associated with measuring soil moisture content and bulk density in real time during soil compaction.

### DETAILED DESCRIPTION

This disclosure relates to a determining soil density and moisture content, which is applicable to any machine that can be used to compact soil for purposes of construction and/or agriculture, among other examples. The machine may be a soil compactor.

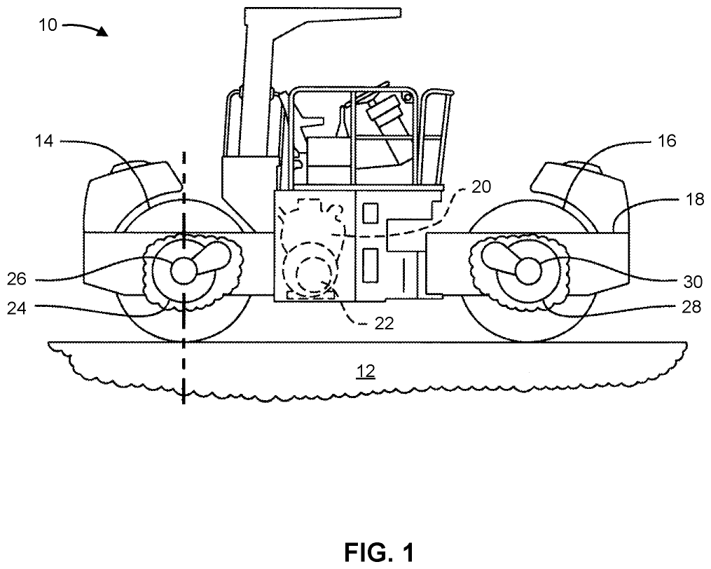

FIG. 1 is a diagram of an example compactor 10 including a system for measuring soil density and moisture content in real time during soil compaction (e.g., a process of compacting soil using the compactor 10).

A compactor 10 may include a machine for increasing density of (i.e., compacting) a compactable material 12, such as soil, gravel, a bituminous mixture, a base layer, an anti-frost layer, asphalt, and/or the like. The compactor 10, for example, can be a double drum vibratory compactor, having a first drum 14 and a second drum 16 rotatably mounted on a main frame 18. The main frame 18 may also support an engine 20 that has an input pump/motor 22 (e.g., a hydraulic pump/motor) connected thereto.

The first drum 14 may include a first vibration component 24 (e.g., one or more unbalance vibrators) that is operatively connected to a first output motor 26 (e.g., a hydraulic motor), while the second drum 16 may include a second vibration component 28 (e.g., one or more unbalance vibrators) that is operatively connected to a second output motor 30. The first drum 14 and the second drum 16 may have more than one vibration component per drum. Further, while compactor 10 is illustrated as a double drum compactor, compactor 10 may be a single drum compactor. An input pump/motor 22 may be included in a vibratory system associated with providing output torque to vibration component 24 and/or vibration component 28.

As indicated above, FIG. 1 is provided as an example. Other examples are possible and may differ from what was described in connection with FIG. 1.

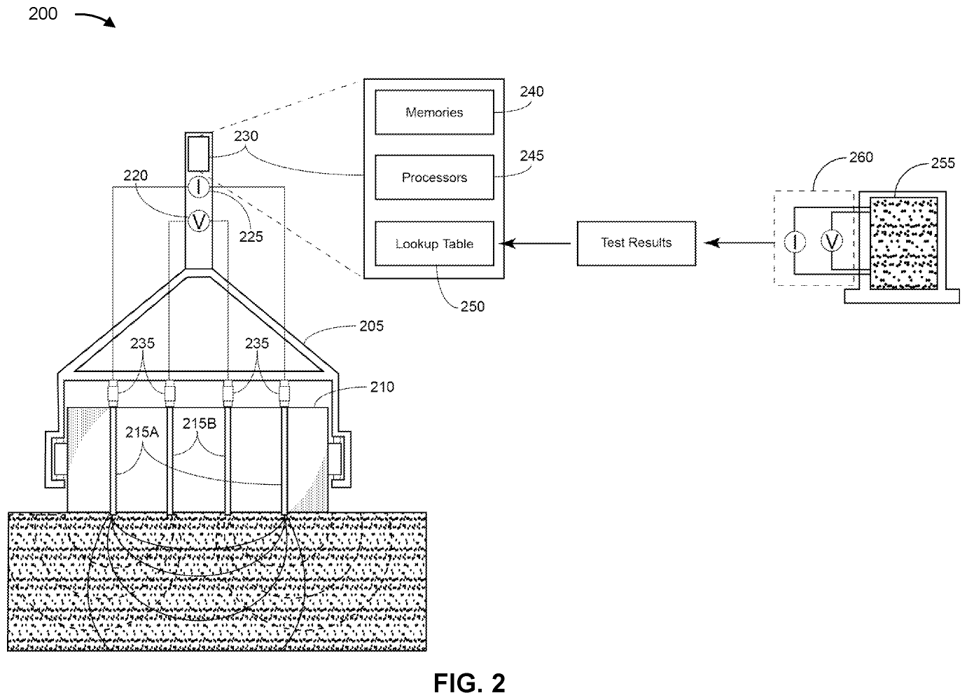

FIG. 2 is a diagram of an example soil measurement device 200 for estimating soil moisture content and bulk density. The soil measurement device 200 may be incorporated into the compactor 10 of FIG. 1. As shown in FIG. 2, the example soil measurement device 200 includes a frame 205, a roller 210, electrodes 215, a voltmeter 220, an ammeter 225, and a controller 230. The soil measurement device 200 may be incorporated into, for example, one of, or both, drums 14, 16 shown and discussed above in FIG. 1. Alternatively, the soil measurement device 200 may be separate from the drums 14, 16 of the compactor 10 of FIG. 1.

The frame 205, which may serve as a structural base of the soil measurement device 200, may be made from metal or plastic. The choice of material for the frame 205 can vary based on desired properties like strength, corrosion resistance, or weight. The frame 205 may be manufactured via a process such as welding, forging, or casting. The frame 205 may be the same as or different from the main frame 18 of FIG. 1.

The roller 210 may be formed from a hard rubber disk operatively connected to the frame 205. Springs 235 may be used to attach the roller 210 to the frame 205 to provide suspension that helps the roller 210 navigate the terrain. Moreover, the springs 235 may be used to electrically connect the electrodes 215 to the voltmeter 220 or ammeter 225. As discussed above, the roller 210 may be one of the drums 14, 16 of the compactor 10 shown in FIG. 1. Alternatively, the roller 210 may be separate from the drums 14, 16 of the compactor 10. When separate from the drums 14, 16, the roller 210 may have a smaller diameter than the drums 14, 16. For example, the roller 210 may have a diameter of about 6 inches.

The electrodes 215 may be disposed on an outer surface of the roller 210. A first pair of electrodes 215A may be used to measure a soil voltage (e.g., a voltage across a first section of soil) and a second pair of electrodes 215B may be used to measure a soil current (e.g., a current flow through a second section of soil). The electrodes 215 may be made from conductive materials like copper or aluminum, and the type of material may be based on factors such as conductivity and resistance to corrosion. The electrodes 215 may output signals to the voltmeter 220 or the ammeter 225, as discussed in greater detail below. Each electrode may be wrapped around the outer surface of the roller 210, and the electrodes 215 may be spaced from one another. Alternatively, the electrodes 215 may be embedded in the outer surface of the roller 210. The electrodes 215 may have a thickness on the order of 0.25 inches. During use of the roller 210 on soil, each of the electrodes 215 may be in contact with the soil. The contact with the soil may be continuous (e.g., each electrode is always in contact with the soil) or periodic (e.g., each electrode contacts the soil at various times depending on the angular rotation of the roller 210). The electrodes 215 may be in contact with the soil at the same time as one another so that the first pair of electrodes 215A and the second pair of electrodes 215B capture soil voltage and soil current, respectively, values across the same sections of soil (e.g., the first section of soil and the second section of soil at least partially overlap).

The voltmeter 220 is configured to measure the soil voltage from signals output by the first pair of electrodes 215A. For example, the voltmeter 220 may be configured to determine the soil voltage of the first section of soil by calculating a difference between the voltage signals output by each of the first pair of electrodes 215A.

The ammeter 225 is configured to measure the soil current. For example, the ammeter 225 may be configured to measure the current flow through the second section of soil using the signals output by the second pair of electrodes 215B.

The controller 230, which may include one or more memories 240 and one or more processors 245, may be configured to receive signals from the voltmeter 220 and the ammeter 225. The controller 230 may be configured to estimate the soil density and moisture content of the soil based on the signals output by the voltmeter 220 and the ammeter 225. For example, the controller 230 may be configured to calculate a resistivity of the soil. The resistivity may be defined as the soil voltage divided by the soil current. The controller 230 may be configured to query a lookup table 250 for the estimated soil density and moisture content. For example, the controller 230 may be configured to query the lookup table 250 based on the resistivity of the soil.

The lookup table 250 may be populated by testing soil samples 255. For example, the soil samples 255 may have a known soil density and moisture content as a result of lab-based soil testing methods, which may include measuring the voltage and current flow across the soil samples 255, using lab testing equipment 260 (e.g., a voltmeter and ammeter different from those included in the compactor 10), and calculating the resistivity of the soil samples 255 (shown as “Test Results” in FIG. 2). The resistivity may be associated with the known soil density and moisture content in the lookup table 250. The lookup table 250 may be stored locally at the controller 230 (e.g., in a memory) or stored in a cloud and accessible via, for example, a wireless network.

The controller 230 may be configured to output an alert signal. The alert signal may include an audible alert (e.g., a siren, beep, or other noise), a visual alert (e.g., illuminating a light), and/or a combination thereof, among other examples. The alert signal may indicate, to an operator of the compactor 10, that the soil has been compacted to the desired soil density and moisture content. Alternatively or in addition, the alert signal may indicate, to the user, that the user should change the compactor type (e.g., switch to a different style of drums than those currently being used), the compactor size (e.g., switch to drums of a different size than those currently being used), a compactor weight (e.g., increase or decrease the weight of the drums), a moisture level (e.g., add moisture to or remove moisture from the soil), or a material level (e.g., add soil to or remove soil from the area).

The controller 230 may be configured to output a control signal to one or more components of the compactor 10. For example, the controller 230 may be configured to output a control signal to control operation of one of, or both, drums 14, 16. The control signal to control operation of one of or both drums 14, 16 may include a drum vibration control signal, a moisture control signal, or a compaction control signal. The drum vibration control signal may be output to the first vibration component 24 to control the vibration of the first drum 14 and/or the second vibration component 28 to control the vibration of the second drum 16. Accordingly, the drum vibration control signal may cause the drums 14, 16 to vibrate in accordance with the control signal output by the controller 230. The moisture control signal may be output to a nozzle controller 230 configured to control the flow of water to the soil. For example, the nozzle controller 230 may be configured to open or close a nozzle in accordance with the moisture control signal. When open, the nozzle may allow water, stored in a reservoir, to spray onto the soil. When closed, the nozzle may prevent water from exiting the reservoir. The compaction control signal may be configured to adjust one or more operating parameters of the compactor 10. For example, the compaction control signal may adjust a speed of the compactor 10.

The controller 230 may be configured to output the alert signal, the control signal, or both, based on the signals output by the voltmeter 220, the ammeter 225, or both, without querying the lookup table 250.

Accordingly, with the soil measurement device 200, the soil density and moisture content may be determined in real time during soil compaction. Moreover, when the desired soil density and moisture content has been obtained through soil compaction, the user of the compactor 10 may be alerted, which can help avoid over-compacting the soil. Further, by outputting one or more of the control signals, the soil measurement device 200 may control one or more components of the compactor 10 to improve the soil compaction process.

As indicated above, FIG. 2 is provided as an example. Other examples may differ from what is described with regard to FIG. 2.

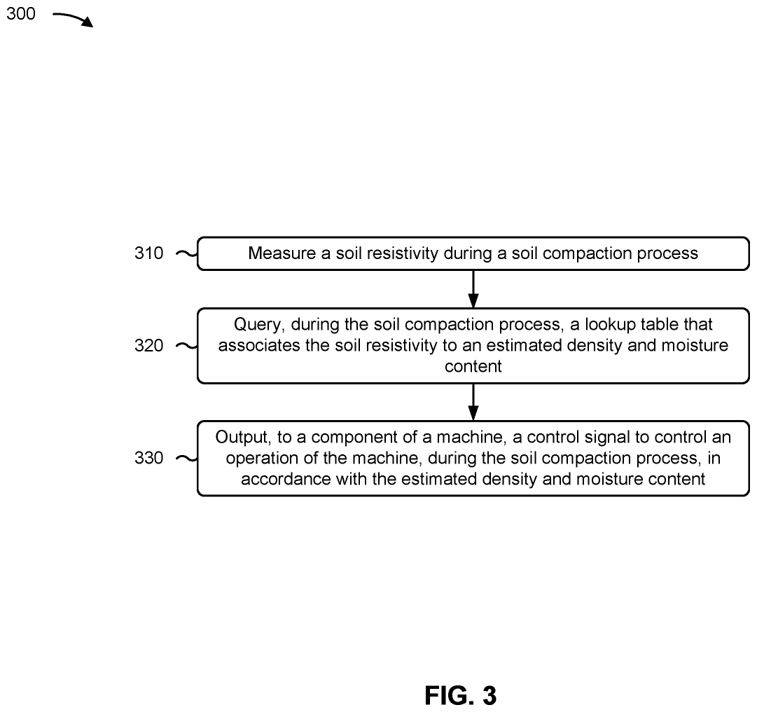

FIG. 3 is a flowchart of an example process 300 associated with measuring soil moisture content and bulk density in real time during soil compaction. One or more process blocks of FIG. 3 may be performed by a soil measurement controller (e.g., soil measurement controller 230). Additionally, or alternatively, one or more process blocks of FIG. 3 may be performed by another device or a group of devices separate from or including the soil measurement controller, such as another device or component that is internal or external to the compactor 10, the soil measurement device 200, and/or a combination thereof, among other examples.

As shown in FIG. 3, process 300 may include measuring a soil resistivity during a soil compaction process (block 310). For example, the soil measurement controller may measure a soil resistivity. Measuring the soil resistivity may include measuring a soil voltage (e.g., a voltage across a section of soil), measuring a soil current (e.g., a current flow through a section of soil), and/or a combination thereof, among other examples. Measuring the soil resistivity may further include calculating the soil resistivity from the soil voltage and the soil current.

As further shown in FIG. 3, process 300 may include querying a lookup table (e.g., lookup table 250) that associates the soil resistivity to an estimated density and moisture content (block 320). For example, the soil measurement controller may query a lookup table that associates the soil resistivity to an estimated density and moisture content, as described above.

As further shown in FIG. 3, process 300 may include outputting, to a component of a machine, a control signal to control an operation of the machine, during the soil compaction process, in accordance with the estimated density and moisture content (block 330). For example, the soil measurement controller may output, to a component of a machine, a control signal to control an operation of the machine in accordance with the estimated density and moisture content, as described above.

The process 300 may include generating the lookup table. Generating the lookup table may include determining a soil resistivity for a plurality of soil samples (e.g., soil samples 255), with each of the plurality of soil samples having a known density and moisture content.

Although FIG. 3 shows example blocks of process 300, in some implementations, process 300 may include additional blocks, fewer blocks, different blocks, or differently arranged blocks than those depicted in FIG. 3. Additionally, or alternatively, two or more of the blocks of process 300 may be performed in parallel.

### INDUSTRIAL APPLICABILITY

With the soil measurement device, which may be used in construction and/or agriculture, among other examples, the soil density and moisture can be measured, in real time, during soil compaction. Because the soil measurement device can be incorporated into the soil compactor, the soil density and moisture can be measured more frequently during the soil compaction process than by using techniques that require specialized equipment or certified technicians. Further, the soil measurement device may output alert signals and/or control signals that can be used to modify the compaction in real-time during the soil compaction process. Accordingly, with the soil measurement device, the compactor may compact the soil more quickly and more accurately, which can reduce costs and result in a higher-quality soil compaction.

The foregoing disclosure provides illustration and description, but is not intended to be exhaustive or to limit the implementations to the precise forms disclosed. Modifications and variations may be made in light of the above disclosure or may be acquired from practice of the implementations. Furthermore, any of the implementations described herein may be combined unless the foregoing disclosure expressly provides a reason that one or more implementations cannot be combined. Even though particular combinations of features are recited in the claims and/or disclosed in the specification, these combinations are not intended to limit the disclosure of various implementations. Although each dependent claim listed below may directly depend on only one claim, the disclosure of various implementations includes each dependent claim in combination with every other claim in the claim set.

When “a processor” or “one or more processors” (or another device or component, such as “a controller” or “one or more controllers”) is described or claimed (within a single claim or across multiple claims) as performing multiple operations or being configured to perform multiple operations, this language is intended to broadly cover a variety of processor architectures and environments. For example, unless explicitly claimed otherwise (e.g., via the use of “first processor” and “second processor” or other language that differentiates processors in the claims), this language is intended to cover a single processor performing or being configured to perform all of the operations, a group of processors collectively performing or being configured to perform all of the operations, a first processor performing or being configured to perform a first operation and a second processor performing or being configured to perform a second operation, or any combination of processors performing or being configured to perform the operations. For example, when a claim has the form “one or more processors configured to: perform X; perform Y; and perform Z,” that claim should be interpreted to mean “one or more processors configured to perform X; one or more (possibly different) processors configured to perform Y; and one or more (also possibly different) processors configured to perform Z.”

As used herein, “a,” “an,” and a “set” are intended to include one or more items, and may be used interchangeably with “one or more.” Further, as used herein, the article “the” is intended to include one or more items referenced in connection with the article “the” and may be used interchangeably with “the one or more.” Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”). Further, spatially relative terms, such as “below,” “lower,” “above,” “upper,” and the like, may be used herein for ease of description to describe one element or feature's relationship to another element(s) or feature(s) as illustrated in the figures. The spatially relative terms are intended to encompass different orientations of the apparatus, device, and/or element in use or operation in addition to the orientation depicted in the figures. The apparatus may be otherwise oriented (rotated 90 degrees or at other orientations) and the spatially relative descriptors used herein may likewise be interpreted accordingly.

What is claimed is:

1. A method, comprising:
measuring a soil resistivity during a soil compaction process; and
querying, during the soil compaction process, a lookup table that associates the soil resistivity to an estimated density and moisture content.

2. The method of claim 1, further comprising outputting, to a component of a machine, a control signal to control an operation of the machine, during the soil compaction process, in accordance with the estimated density and moisture content.

3. The method of claim 1, wherein measuring the soil resistivity includes measuring a soil voltage.

4. The method of claim 3, wherein measuring the soil resistivity includes measuring a soil current.

5. The method of claim 4, wherein measuring the soil resistivity includes calculating the soil resistivity from the soil voltage and the soil current.

6. The method of claim 1, further comprising generating the lookup table.

7. The method of claim 6, wherein generating the lookup table includes determining a soil resistivity for a plurality of soil samples, each of the plurality of soil samples having a known density and moisture content.

8. A soil compaction measurement controller, comprising:
one or more memories; and
one or more processors, communicatively coupled to the one or more memories, configured to:
determine a soil resistivity;
query a lookup table associating the soil resistivity to an estimated density and moisture content, the lookup table being stored in the one or more memories; and
output, to a component of a machine, a control signal to control an operation of the machine in accordance with the estimated density and moisture content.

9. The soil compaction measurement controller of claim 8, wherein the soil resistivity is based, at least in part, on one or more of a soil voltage measurement or a soil current measurement.

10. The soil compaction measurement controller of claim 8, wherein the control signal includes one or more of a drum vibration control signal, a moisture control signal, or a compaction control signal.

11. The soil compaction measurement controller of claim 8, wherein the one or more processors are further configured to output an alert signal.

12. The soil compaction measurement controller of claim 11, wherein the alert signal indicates an alert to change one or more of a compactor type, a compactor size, a compactor weight, a moisture level, or a material level.

13. A soil compactor machine, comprising:
a drum;
a plurality of electrodes disposed on the drum;
a voltmeter electrically connected to at least two of the plurality of electrodes and configured to output a soil voltage measurement;
an ammeter electrically connected to at least two of the plurality of electrodes and configured to output a soil current measurement; and
a soil compaction measurement controller in communication with the voltmeter and the ammeter, the soil compaction measurement controller being configured to:
receive the soil voltage measurement and the soil current measurement;
determine an estimated density and moisture content from the soil voltage measurement and the soil current measurement; and
control operation of the drum in accordance with the estimated density and moisture content.

14. The soil compactor machine of claim 13, wherein the soil compaction measurement controller is configured to determine the estimated density and moisture content by querying a lookup table.

15. The soil compactor machine of claim 13, wherein the soil compaction measurement controller is configured to determine the estimated density and moisture content by calculating a soil resistivity using the soil voltage measurement and the soil current measurement.

16. The soil compactor machine of claim 13, wherein the soil compaction measurement controller is configured to control operation of the drum by outputting a control signal.

17. The soil compactor machine of claim 16, wherein the control signal includes one or more of a drum vibration control signal, a moisture control signal, or a compaction control signal.

18. The soil compactor machine of claim 13, further comprising a vibration component configured to vibrate the drum.

19. The soil compactor machine of claim 18, wherein the vibration component is configured to vibrate in accordance with a control signal output by the soil compaction measurement controller.

20. The soil compactor machine of claim 13, wherein the soil compaction measurement controller is configured to output an alert signal for alerting a user to change one or more of a compactor type, a compactor size, a compactor weight, a moisture level, or a material level.
